(19) United States
(12) Patent Application Publication
(10) Pub. No.: US 2018/0193002 A1
(43) Pub. Date: Jul. 12, 2018

(54) CAT-PAN LITTER-LESS LINER PRODUCT AND METHOD

(71) Applicant: Lora Langefels, New Orleans, LA (US)

(72) Inventor: Lora Langefels, New Orleans, LA (US)

(21) Appl. No.: 15/401,785

(22) Filed: Jan. 9, 2017

(51) Int. Cl. A61B 10/00; A01K 1/01; A01K 29/00

(52) U.S. Cl. CPC A61B 10/0038; A61B 2503/40; A01K 29/005; A01K 1/011

(57) ABSTRACT

A cat-box litter-less liner product and method for managing cat excreta, in a cat box, without litter, comprising an impermeable liner with an affixed absorbent pad, which fits into and over the sides of a cat box in use, and forms a disposable bag after use, providing the cat with a place to urinate and defecate, without the spilling of any waste or other substance from the cat box during use or when removed and disposed of. This invention solves problems associated with the use of kitty litter in a cat pan, and provides a convenient and sanitary way to obtain stool and urine samples for health evaluation and analysis, and to regularly and periodically make quick visual inspections of the cat's excreta for early detection of health problems.

### BACKGROUND OF THE INVENTION

This invention is a cat-pan litter-less liner product and method for managing cat excreta, in a cat pan, without litter, comprising an impermeable liner with an affixed absorbent pad, which fits into and over the sides of a cat pan in use, and forms a disposable bag after use, providing the cat with a place to urinate and defecate, without the spilling of any waste or other substance from the cat pan during use or when removed and disposed of.

The use of kitty litter in a cat pan, which is presently the most commonly used method of management, comes with several disadvantages. Packages of the litter are bulky and heavy, and have to be purchased and carried often. The litter is made of fine granules, which can easily become airborne. When used in a cat pan, the litter tends to be scattered or spilled outside the cat pan, by the cat's movements and by other factors. The cat's excreta is also scattered and spilled outside the pan. Scooping used litter for disposal stirs it up and further causes scattering or spilling of both litter and cat excreta. The resulting used litter is difficult to dispose of, as it may clog plumbing, and the process of disposal tends to further scatter or spill the litter and excreta. Even the process of pouring new litter to the cat pan can lead to scattering and spillage.

Another disadvantage of kitty litter is that it contaminates and ruins the ability to examine or analyze the cat's excreta for veterinary health purposes. Cat owners are often instructed to examine the urine or fecal sample of the pet to ensure that the pet remains healthy after a visit to the veterinarian. At home, regular visual checking of the cat's excreta, such as for the presence of blood or worms, is a good idea, but contamination from kitty litter interferes with the ability to do this checking. In a veterinary setting, a veterinarian typically has to catheterize the cat to retrieve a urine sample, inserting a needle into the bladder of the cat.

A number of issued patents disclose the use of a multi-layered liner meant to be placed in a cat pan, with ends that may stretch over the lid of the cat pan for easy application and removal. Additionally, there are several patents that disclose the use of an odor eliminator and/or attractive scent meant to attract the cat to the pan. However, none of the references disclose litter-less liner product and method for managing cat waste, in a cat box, without litter, comprising an impermeable liner with an affixed absorbent pad, which fits into and over the sides of a cat box in use, and forms a disposable bag after use, as reviewed in detail below.

U.S. Pat. No. 4,869,204 was issued Sep. 26, 1989 to Patrick Yananton for an “Odorless Animal Litter Unit Having Securing Means.” The claw-resistant sorbent pad laminate is employed for the collection of animal urine. The laminate of the Yananton unit includes a bottom sheet layer of moisture-impermeable material, an intermediate sorbent layer of material having a high sorbency capacity for urine, and a top claw resistant screen means. The laminate is secured to a container by means of securing means around the periphery of the bottom sheet layer. In one embodiment of the invention, the securing means is an elasticized area or areas around the periphery of the moisture-impermeable material. In an alternate embodiment, a drawstring is movably affixed to the periphery of the bottom sheet layer.

U.S. Pat. No. 4,836,141, issued Jun. 6, 1989 to Roxana R. Whitfield for an “Odor Control Pet Excrement Pan,” covers an odor-controlling pet excrement container having a substantially flat pan. In the pan, a conforming protective liner of flexible sheet material is removably mounted, the liner having a layer of absorptive material and being adapted to contain a layer of litter material if desired. After use, the liner and its contents are removed from the pan and the excrement and litter are sealed within the liner by means of a drawstring. Super absorbent material capable of gelling liquid urine and suitable odor suppressants are incorporated in the liner and its contents.

U.S. Pat. No. 5,850,798 was issued Dec. 22, 1998 to Eric M. Engel for a “cat-pan Liner.” The invention covers a liner for a cat pan having sides extending upwardly from a bottom to a vertical height. The liner comprises a bag for placement in the cat pan. The bag comprises a bottom portion for lining the cat pan, and a contiguous top portion. A fluid-impermeable layer is placed within the box, located within the bottom portion of the bag and bonded to the bag. A sorbent layer is placed above the fluid-impermeable layer. A clay-resistant layer is placed above the sorbent layer and affixed to the fluid-impermeable layer so as to stiffen the affixed layers, whereby they tend to stay in place in the cat pan. The top portion of the bag drapes over the sides of the box for a drape length and provides ballast for helping the liner to stay in place.

U.S. Pat. No. 4,919,078, issued Apr. 24, 1990 to Glenn E. Morrison for a “Litter Packaging System,” discloses a pet litter packaging apparatus that includes a rectilinear cardboard container whose flaps expand to form a litter box having an interior of sufficient size to accommodate a pet. Litter bags lie on their sides with their openings directed against a vertical door. Beams are used to hold the bags open and to support the flaps in a house-like configuration. The pet eliminates within the bags, which can be disposed of one after the other without the pet owner having to transfer the litter.

U.S. Pat. No. 4,858,559, issued Aug. 22, 1989 to Pamela J. Allen for a “Disposable Pet Toilet Unit,” covers a disposable pet toilet unit having a body portion within which a deodorizing, absorbent, granular material is contained for use by household pets to defecate and urinate. The body portion is made of a moisture-proof material and includes a bottom and a plurality of sides extending upwardly from the bottom to form an opening. A moisture-proof film is secured to the body portion and gatherable about the opening thereof by a closure, such as a drawstring. Thus, when the deodorizing, absorbent, granular material becomes sufficiently soiled so as to no longer adequately deodorize and absorb odor and moisture, the drawstring may be pulled to gather the film together about the opening to substantially seal it. In this manner, the disposable pet toilet unit may be transported and discarded without spillage or leakage of the soiled, deodorizing, absorbent, granular material.

U.S. Pat. No. 4,813,374 was issued Mar. 21, 1989 to Claude R. Sides for a “Prepackaged Liner and Litter Assembly.” The assembly for animal waste includes a bag with a plurality of liner and litter units within the bag. Each liner and litter unit includes a liner of flexible material with the litter contained therein. Within the bag, each liner is configured in pouch configuration to facilitate holding the litter therein, and each liner is adapted to being opened at the top and arranged within a litter box to extend over the bottom and sides of the box. The liner and litter units are arranged within the bag with the tops of the liners toward the top of the bag.

U.S. Pat. No. 4,807,563 was issued Feb. 28, 1989 to Ronald L. Berry and Linda J. Scholes-Witty on a “Self-Contained, Disposable, Sanitary Toilet Facility for Pets.” The facility covers a pet litter box, including a device for locking the box after it has been used and re-closed, formed from a single foldable blank. When the box is closed or re-closed, it has inter-laying first and second halves, each having bottom and sidewall portions. The halves are connected to one another by a joining portion, and joining sidewalls. Each of the sidewalls of the first half carries a tab. The sidewall portions of the second half each define a diagonal crease, and the pair of joining side-wall portions each define a diagonal crease and a pair of vertical creases. When the box is fully open its first, second, and joining portions define an open tray. After the system has been used, the box can be re-closed and locked. During re-closing, the joining sidewall portions and the two sidewalls of the second half all fold inwardly at their creases, thereby forming a pair of slots between the pairs of creases on each side. Then, by inserting the tabs carried by the first half sidewalls into the slots which are formed between the creases, re-closing and locking of the box is completed. The box blank may include a flap for securing the box closed before it is utilized, and a handle, and may also include all items which are necessary for pet toiletry, such as a bag of litter, and a liner sized to fit over the open tray.

U.S. Pat. No. 4,756,273, issued Jul. 12, 1988 to Patrick Yananton, covers a “Reversible Bag for Use with Litter Bag.” In this second Yananton invention, a disposable animal litter pad is combined with a moisture-impermeable bag for easy disposal. The litter pad includes a sorbent pad laminate for the collection of animal urine and a top claw-resistant screen made of a urine-permeable, flexible member of material substantially inert to urine. The disposable animal litter pad laminate has a bottom layer of two sheets of moisture-impermeable material peripherally sealed together along three sides to form a bag-like structure. The sorbent pad laminate also includes a sorbent layer of material, positioned adjacent the moisture-impermeable material, and having a high sorbtion capacity for urine. Disposal is accomplished by reversing the bag over the litter pad, thereby enclosing the litter pad, along with the animal waste and litter and facilitating disposal.

U.S. Pat. No. 4,819,580 was issued on Apr. 11, 1989 to David A. Foldes for a “Disposable Cat Litter Box.” The open-top receptacle is embraced with a tube of waterproof polymer film material. The tube extends beyond the receptacle and is sufficiently long and large so that when it is desired to dispose of the litter box, the tube is inverted over the entire litter box to enclose and seal it. The disposable cat litter box comprises (1) a receptacle having a bottom and having a top edge mounted on the bottom and extending above the bottom, with the top edge defining an interior space within the receptacle, which is open at the top; and (2) a flexible waterproof tube, the tube having a portion that is sufficiently large to receive therein the receptacle, and being sufficiently large so that the tube portion can extend below the top edge of the receptacle to form a recess within and below the top edge of the receptacle to receive litter; where the tube has a closed end thereon and the receptacle being positioned within the tube portion adjacent the closed end of the tube; and where the tube extends away from the receptacle to an open end, the tube having an intermediate closure between the portion of the tube which contains the receptacle and the open end, the tube being sufficiently long from the intermediate closure so that the open end of the tube can be inverted over the portion of the tube that contains the receptacle to enclose the receptacle and the recess in the receptacle formed by the tube.

U.S. Pat. No. 4,840,140 was issued on Jun. 20, 1989 to Patrick Yananton and Janice P. Parker for a “Securing Means for [an] Odorless Animal Litter Unit.” The invention covers a combination of a litter device for use with cats, which has a sorbent pad laminate for the collection of animal urine. The device consists of a bottom sheet layer of moisture-impermeable material in contact with the base and walls of the litter device, an intermediate sorbent layer of material having a high absorbency capacity for urine, a top claw-resistant screen, and securing device. The securing device removably affixes the sorbent pad laminate to the litter device, thereby preventing the relative movement between the sorbent pad and the litter device that can be caused by the clawing action of an animal.

U.S. Pat. No. 5,526,770, issued on Jun. 18, 1996 to Theodore M. Kiebke for a “Biodegradable Dustless Cat Litter,” covers a dustless cat litter that includes a wetting additive to agglomerate dust to litter ingredients and promote hydration. Exemplary wetting additives are water soluble, anionic, amphoteric or synthetic surfactants, surfactant mixtures, soaps, or synthetic detergents, which are applied to the litter ingredients to bind dust and fines contained in the mixtures. A masking fragrance, neutralizer, or attractant may be admixed with the surfactant. Exemplary hydrating cat litter or additive mixtures, which solidify upon contact with liquids to encapsulate pet waste matter, include a granulated base material (e.g., whole or coarse-ground agricultural grains, pulse crops, clay granules, or mixtures thereof), a gluten-containing material (e.g., semolina or ground wheat), and may include sodium bicarbonate and/or a carrier bound fragrance. A quantity of surfactant on the order of ½ to 7 ounces per 10 pounds litter containing 5% to 10% fines is applied to the ingredients during blending of the mixture.

### SUMMARY OF THE INVENTION

This invention provides a cat-box litter-less liner product and method for managing cat waste, in a cat box, without litter, comprising an impermeable liner with an affixed absorbent pad, which fits into and over the sides of a cat box in use, and forms a disposable bag after use, providing the cat with a place to urinate and defecate, without the spilling of any waste or other substance from the cat box during use or when removed and disposed of.

This invention solves problems associated with the use of kitty litter in a cat pan, and provides a convenient and sanitary way to obtain stool and urine samples for health evaluation and analysis, and to regularly and periodically make quick visual inspections of the cat's excreta for early detection of health problems.

### BRIEF DESCRIPTION OF DRAWINGS

Reference will now be made to the drawings, wherein like parts are designated by like numerals, and wherein:

FIG. 1 is a perspective schematic view of a first embodiment of the cat-pan litter-less liner of the invention.

FIG. 2 is an exploded schematic view of a first embodiment of the cat-pan litter-less liner of the invention.

FIG. 3 is a perspective schematic view of a second embodiment of the cat-pan litter-less liner of the invention; and

FIG. 4 is an exploded schematic view of a second embodiment of the cat-pan litter-less liner of the invention.

### DETAILED DESCRIPTION OF THE DRAWINGS

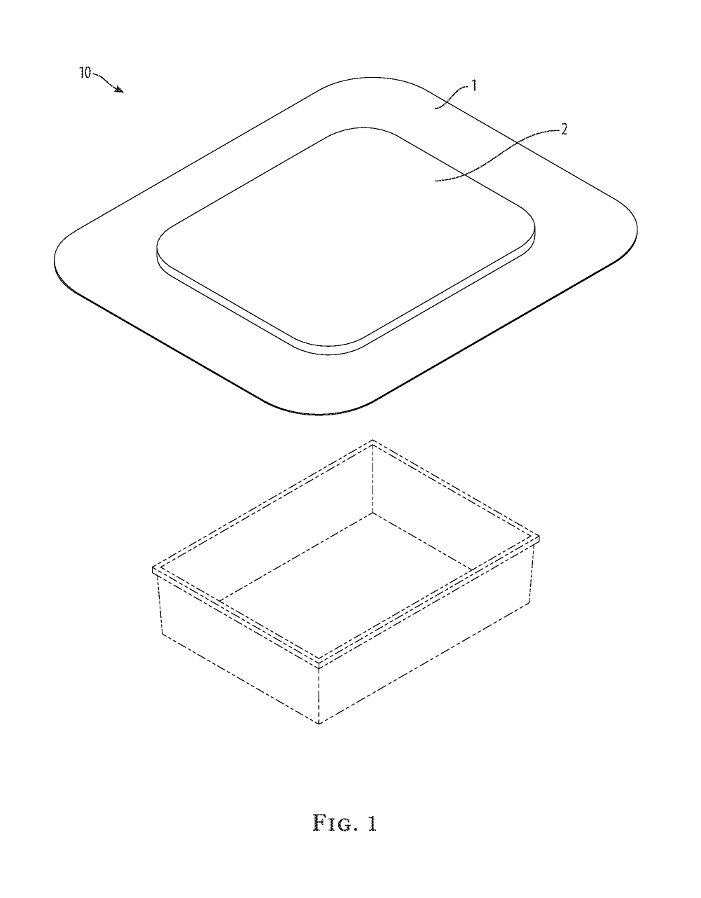

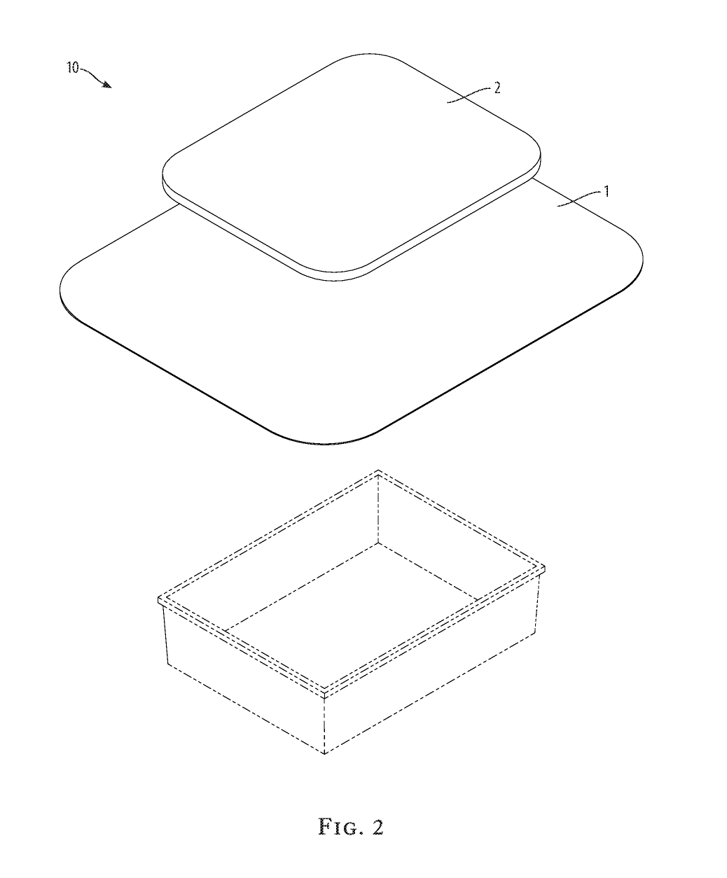

Referring to FIG. 1 & FIG. 2, showing a first embodiment of the cat-pan litter-less liner 10 of the invention, the cat-pad litter-less liner 10 is meant to be placed into a standard cat pan, which is generally between one and two feet in each lateral dimension, covering an area of approximately two to three square feet, and a depth of from two to five inches. The cat-pan litter-less liner 10 completely replaces and eliminates the use of kitty litter, which eliminates the purchase and handling of litter, the dust of litter, the scattering of litter and waste, and the contamination of cat excreta making it useless for health analysis.

The cat-pan litter-less liner 10 provides an impermeable liner 1 which fits into and over the sides of a cat pan. The impermeable liner 1 contains the cat waste, and forms a disposable bag after use. The impermeable liner 1 can have an elastic edge for gripping the rim of the cat pan. The impermeable liner 1 can be made from a sheet of rubber or plastic or of rubberized or plasticized paper or textile material.

Affixed to the center of the upward face or inner face of the impermeable liner 1 is an absorbent pad 2. The absorbent pad 2 absorbs cat excreta, which keeps the excreta from scattering. The absorbent pad 2 does not contaminate nor denature the cat excreta, which can be easily and conveniently be given a spot analysis when removing the cat-pan litter-less liner 10 after the cat has used it. The absorbent pad 2 is meant to be disposed of after use, contained within the disposable bag formed by the impermeable liner 1.

In use, the cat-pan litter-less liner 10 is placed to line the bottom and sides of the cat pan, providing the cat with a place to urinate and defecate. The cat-pan litter-less liner 10 prevents spilling of any excreta or other substance from the cat pan. Periodically, the used cat-pan litter-less liner 10 is removed and either disposed of or kept for analysis. The cat-pan litter-less liner 10 can be removed and disposed of without spilling any excreta or other substance from the cat pan. During the removal and disposal process the cat excreta can be visually spot-checked for blood, parasites, or other indications of poor health.

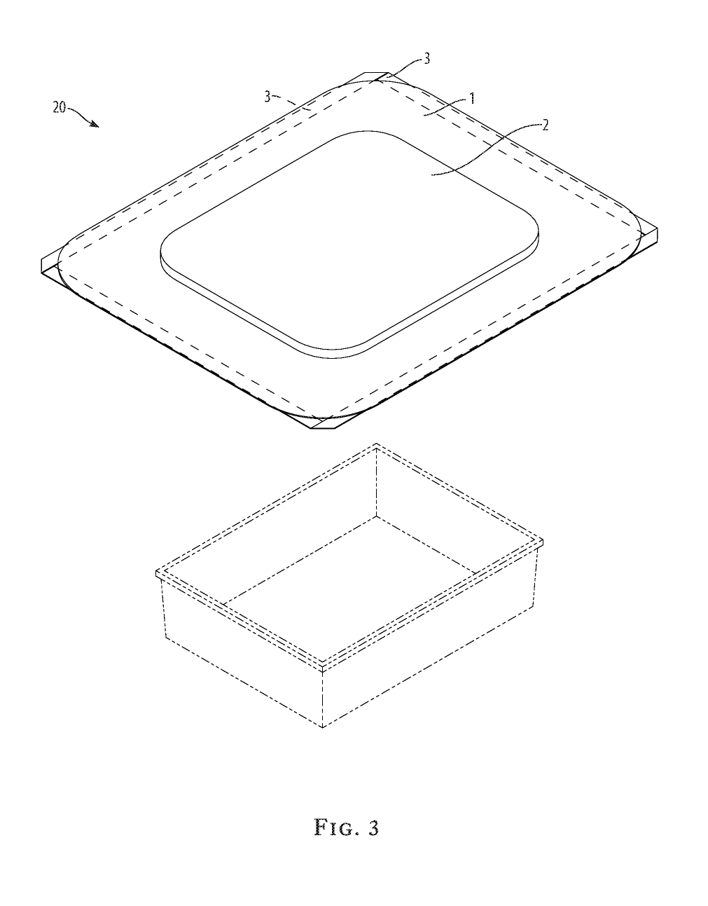

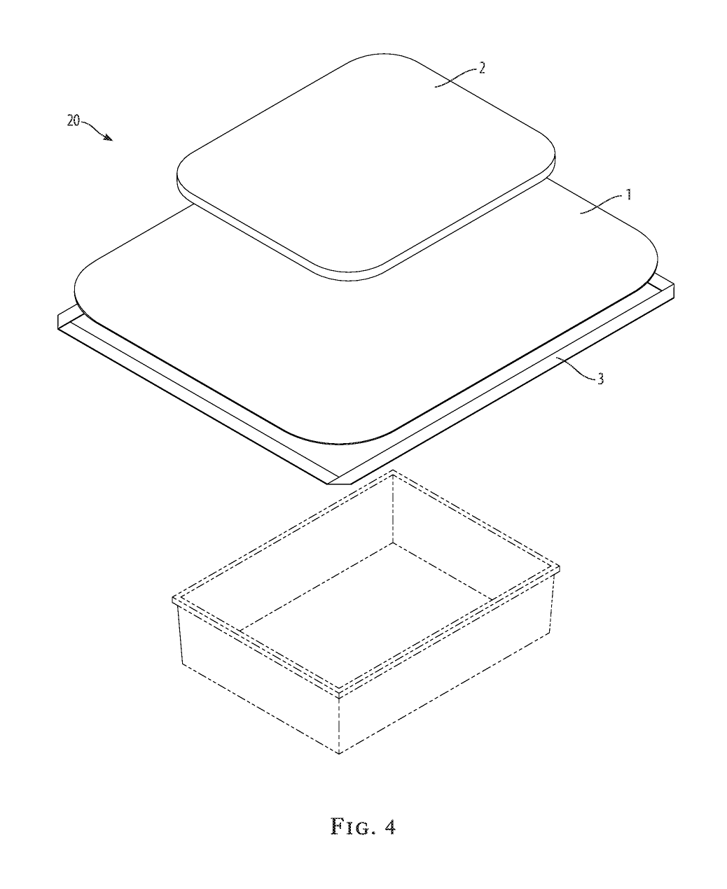

Referring to FIG. 3 & FIG. 4, showing a second embodiment of the cat-pan litter-less liner 10 of the invention, the cat-pan litter-less liner 10 can be provided with a strap or band 3 around the perimeter of the impermeable liner 1. Such a band 3 can be used as a tie strap when a used cat-pan litter-less liner 10 is being disposed of. It can also be used to gather the perimeter of the impermeable liner 1 about the rim of the cat pan. The band 3 can be made of a similar material to the impermeable liner 1. The band 3 can also be made of an elastic material that will gather the cat-pan litter-less liner 10 about the rim of the cat pan.

The impermeable liner 1 or the absorbent pad 2 of the cat-pan litter-less liner 10 may also feature an applied or incorporated substance that is attractive to cats. The absorbent pad 2 of the cat-pan litter-less liner 10 can have an applied or incorporated substance that neutralizes odors without denaturing or contaminating the cat excreta. The absorbent pad 2 of the cat-pan litter-less liner 10 can have an applied or incorporated substance that reacts with cat excreta in such a way as to provide a visual indication of the presence of blood or other marker of poor health.

Many changes and modifications can be made in the present invention without departing from the spirit thereof. I therefore pray that my rights to the present invention be limited only by the scope of the appended claims.

I claim:

1. A cat-pan litter-less liner product for managing cat excreta, in a cat pan, without litter, the cat-pan litter-less liner comprising:
(i) an impermeable liner adapted to fit into and over the sides of a cat pan in use, and further adapted to form a disposable bag after use, having an inner face and an outer face; and
(ii) an absorbent pad affixed to said impermeable liner on the inner face, adapted to absorb cat excreta during use, and further adapted to be disposed of after use;
where, in use, said impermeable liner with said absorbent pad is placed to line the bottom and sides of the cat pan, providing the cat with a place to urinate and defecate;
where, in use, said impermeable liner with said absorbent pad prevents spilling of any excreta or other substance from the cat pan; and
where, in use, the disposable bag formed from said impermeable liner with said absorbent pad is removed and disposed of without spilling any excreta or other substance from the cat pan.

2. The cat-pan litter-less liner product of claim 1, further comprising a band around the perimeter of said impermeable liner.

3. The cat-pan litter-less liner product of claim 1, further adapted to allow recovery of cat excreta without degrading its utility for health analysis.

4. The cat-pan litter-less liner product of claim 1, further comprising a cat attractant incorporated into said impermeable liner.

5. The cat-pan litter-less liner product of claim 1, further comprising a cat attractant incorporated into said absorbent pad.

6. The cat-pan litter-less liner product of claim 1, further comprising an odor-eliminating substance incorporated into said absorbent pad.

7. The cat-pan litter-less liner product of claim 1, further comprising a substance that reacts with cat excreta in such a way as to provide a visual indication of the presence of blood or other marker of poor health, incorporated into said absorbent pad.

8. The cat-pan litter-less liner product of claim 1, further comprising a band around the perimeter of said impermeable liner adapted to be used as a tie strap for disposal of said cat-pan litter-less liner.

9. The cat-pan litter-less liner product of claim 1, further comprising a band around the perimeter of said impermeable liner adapted to be used for gathering of the perimeter of said impermeable liner about the rim of a cat pan.

10. The cat-pan litter-less liner product of claim 1, where said cat-pan litter-less liner is further adapted for the collection of specimens of cat waste for veterinary analysis.

11. A cat-pan litter-less liner method for managing cat excreta, in a cat pan, without litter, comprising:
(i) providing a cat-pan litter-less liner, comprising:
(a) an impermeable liner adapted to fit into and over the sides of a cat pan in use, and further adapted to form a disposable bag after use, having an inner face and an outer face; and
(b) an absorbent pad affixed to said impermeable liner on the inner face, adapted to absorb cat excreta in use, and further adapted to be disposed of after use;
(ii) placing said cat-pan litter-less liner to line the bottom and sides of the cat pan;
(iii) providing the cat with a place to urinate and defecate;
(iv) preventing spilling of any excreta or other substance from the cat pan;
(v) removing said cat-pan litter-less liner from said cat pan without spilling any excreta or other substance from the cat pan; and
(vi) disposing of said cat-pan litter-less liner as a disposable bag without spilling any excreta or other substance.

12. The cat-pan litter-less liner method of claim 11, further comprising a band around the perimeter of said impermeable liner.

13. The cat-pan litter-less liner method of claim 11, further adapted to allow recovery of cat excreta without degrading its utility for health analysis.

14. The cat-pan litter-less liner method of claim 11, further comprising a cat attractant incorporated into said impermeable liner.

15. The cat-pan litter-less liner method of claim 11, further comprising a cat attractant incorporated into said absorbent pad.

16. The cat-pan litter-less liner method of claim 11, further comprising an odor-eliminating substance incorporated into said absorbent pad.

17. The cat-pan litter-less liner method of claim 11, further comprising a substance that reacts with cat excreta in such a way as to provide a visual indication of the presence of blood or other marker of poor health, incorporated into said absorbent pad.

18. The cat-pan litter-less liner method of claim 11, further comprising a band around the perimeter of said impermeable liner adapted to be used as a tie strap for disposal of said cat-pan litter-less liner.

19. The cat-pan litter-less liner method of claim 11, further comprising a band around the perimeter of said impermeable liner adapted to be used for gathering of the perimeter of said impermeable liner about the rim of a cat pan.

20. The cat-pan litter-less liner method of claim 11, where said cat-pan litter-less liner is further adapted for the collection of specimens of cat waste for veterinary analysis.
